(19) United States
(12) Patent Application Publication
(10) Pub. No.: US 2014/0239249 A1
(43) Pub. Date: Aug. 28, 2014

(54) RAPID BIOLOGICAL SYNTHESIS PROCESS TO PRODUCE SEMICONDUCTING CHALCOGENIDE NANOSTRUCTURES FOR TRANSISTOR OR SOLAR CELL APPLICATIONS

(71) Applicant: University of Southern California, Los Angeles, CA (US)

(72) Inventors: Ian R. McFarlane, Los Angeles, CA (US); Mohamed Y. El-Naggar, Pasadena, CA (US)

(73) Assignee: University of Southern California, Los Angeles, CA (US)

(21) Appl. No.: 14/193,769

(22) Filed: Feb. 28, 2014

Related U.S. Application Data
(60) Provisional application No. 61/770,483, filed on Feb. 28, 2013.

(51) Int. Cl. H01L 21/02; C01G 28/00; C12P 3/00; H01L 29/06; H01L 31/0352

(52) U.S. Cl. CPC H01L 21/02606; H01L 29/0669; H01L 31/035227; C12P 3/00; C01G 28/008
USPC 257/9; 136/255; 435/168; 423/561.1

(57) ABSTRACT

The process disclosed herein produces macroscopic quantities of semiconducting arsenic sulfide nanofibers within one to three days. The process is biotically influenced by the bacteria Shewanella sp. Strain ANA-3. The fibers are semiconductors with bandgaps between 2.2 and 2.5 eV. Newly measured semiconducting and bandgap properties can lead to applications in the semiconductor, transistor, and solar energy fields. A faster and more robust biological component makes the overall process more commercially feasible than it would have been otherwise. The faster rate allows for larger yields of nanofibers in a predetermined period of time.

### RELATED APPLICATIONS

The present disclosure claims priority to U.S. Provisional Application 61/770,483, filed on Feb. 28, 2013, which is hereby incorporated by reference in its entirety.

### FIELD

The present disclosure relates generally to semiconducting nanofibers, and more particularly, to a process of producing macroscopic quantities of arsenic sulfide nanofibers within one to three days using the Shewanella sp. strain ANA-3.

### BACKGROUND

The synthesis of nanomaterials by biological or biomimetic means in physiological conditions offers multiple advantages over traditional physical and chemical strategies that typically require more extreme environments (temperature, pressure, and pH). In addition to the promise of cheaper and greener synthesis processes, the resulting biogenic materials can exhibit unique morphologies and physical/chemical properties stemming from the tight control organisms exert over the composition, nucleation, crystallography, and desired function of these materials

U.S. Patent Application Publication No. 2009/0155876 A1 to Hor-Gil Hur et al. (“Hur”) discloses a biological method for preparing arsenic sulfide (As—S) compounds. More particularly, Hur provides a method for production of nanotubes based on As—S compounds including As₂S₃ by reacting thiosulfate S₂O₃²⁻ with arsenate As(V) through mediation of Shewanella sp. strain. However, the process of Hur is slow, and relies on the bacterium Shewanella sp. strain HN-41. Further, it is unclear if the structures produced by the described process are semiconductors. There is thus a need for a process to rapidly produce As—S compounds for applications such as semiconductors.

### SUMMARY

The process disclosed herein produces macroscopic quantities of semiconducting arsenic sulfide nanofibers within one to three days. The process is biotically influenced by the bacteria Shewanella sp. strain ANA-3. The resulting nanofibers are semiconductors with bandgaps between 2.2 and 2.5 eV.

The measured semiconducting and bandgap properties from the resulting nanofibers may lead to applications in the semiconductor, transistor, and solar energy industries, or similar industries. A faster manufactured and more robust biological component makes the overall process of nanofiber production more commercially feasible than it would have been otherwise. The faster rate of growth allows for easier research and development as well as larger yield per day of the As—S compounds.

### BRIEF DESCRIPTION OF THE DRAWINGS

FIG. 1A is a cartoon showing the flow of electrons from an electron donor to the cell to an electron acceptor according to an embodiment.

FIG. 1B illustrates the redox potentials of various redox couples according to an embodiment.

FIG. 2A illustrates precipitated arsenic sulfide after several days according to an embodiment.

FIG. 2B illustrates an SEM image of arsenic sulfide according to an embodiment.

FIG. 2C illustrates arsenic sulfide as a mesh of polydisperse fibers according to an embodiment.

FIG. 3A illustrates an SEM image of a fiber used for the EDS composition measurement according to an embodiment.

FIG. 3B illustrates EDS data showing the fiber to be made of arsenic sulfide according to an embodiment.

FIG. 4 illustrates novel morphologies that cannot be easily duplicated using known abiotic routes according to an embodiment.

FIG. 5A is an SEM image of a fiber with branches coming off at similar angles according to an embodiment.

FIG. 5B illustrates batch XRD data showing crystalline peaks matching those of β-As₄S₄, also referred to as As₈S₈, according to an embodiment.

FIG. 6 is a “Tauc Plot” of washed AsₓS_{y} fibers according to an embodiment.

FIG. 7 illustrates a map of the arr operon responsible for respiring arsenic and the ars operon responsible for detoxifying arsenic inside the cell according to an embodiment.

FIGS. 8A and 8B show bottles containing dead cells and reduced sulfur when injected with arsenic precursors according to an embodiment.

FIG. 9 illustrates an OD reading of two heat killed cultures and one control according to an embodiment.

FIG. 10A illustrates average fiber growth rate versus time according to an embodiment.

FIG. 10B illustrates time-lapse snapshots of extending fibers according to an embodiment.

FIG. 10C illustrates NanoOrange protein stain of a cluster of As—S fibers according to an embodiment.

FIG. 11 illustrates growth curves of four ANA-3 strains according to an embodiment.

FIG. 12 illustrates filtered and unfiltered bottles producing the AsₓS_{y} according to an embodiment.

FIG. 13A is a diagram of a typical MOSFET.

FIG. 13B is a diagram of the As—S nanofiber transistor according to an embodiment.

FIG. 14A illustrates a nanofiber before contact deposition according to an embodiment.

FIG. 14B illustrates a nanofiber after contact deposition according to an embodiment.

FIG. 15 is an SEM image of interleaved gold contacts with deposited As—S fibers according to an embodiment.

FIGS. 16A-B illustrate the properties of a p-type device according to an embodiment.

FIGS. 17A-B illustrate the properties of an n-type device according to an embodiment.

FIGS. 18A-B illustrate the properties of a p- and n-type device according to an embodiment.

### DETAILED DESCRIPTION

A rapid biological synthesis process to produce semiconducting chalcogenide nanostructures for transistor or solar cell applications is described. In the following description, for purposes of explanation, numerous specific details are set forth in order to provide a thorough understanding of the exemplary embodiments. It is apparent to one skilled in the art, however, that embodiments can be practiced without these specific details or with an equivalent arrangement. In some instances, well-known structures and devices are shown in block diagram form in order to avoid unnecessarily obscuring the embodiments.

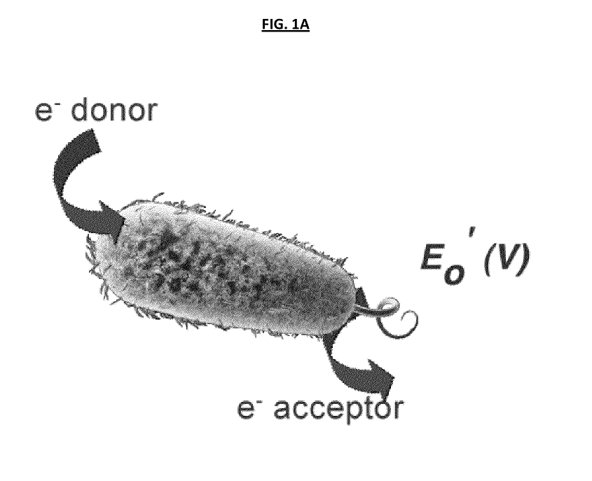

Electron flow is fundamental to biology: organisms extract electrons from a wide array of electron donors (fuels) and transfer them to electron acceptors (oxidants). These redox reactions between electron donors and electron acceptors power the production of ATP. FIG. 1A is a cartoon showing the flow of electrons from an electron donor to the cell to an electron acceptor according to an embodiment. FIG. 1B illustrates the redox potentials of various redox couples. Electron flow for various types of metabolism is shown by the arrows. The acceptor energy minus donor energy gives the maximum energy available from the reaction.

Many prokaryotes can use multiple electron acceptors. Shewanella species, for instance, respire O₂, Mn(IV)/Mn(III)/Fe(III) oxides, NO₃⁻/NO₂⁻, U(VI), Cr(VI), As(V), S₂O₃²⁻, SeO₃²⁻ and TeO₃²⁻. Shewanella's ability to reduce these chalcogenide compounds with the reduced products precipitating in solution, offers a low temperature route to synthesizing chalcogenide nanomaterials in mild environmental conditions (pressure, temperature, and pH), while taking advantage of the exceptional control organisms exert over the structures and functional properties of biogenic materials.

Chalcogenides (materials containing S, Se or Te) are used extensively for their photonic and electrical properties. CdTe and CdSe are important semiconductors used in solar cells and luminescent quantum dots. Photodeformation has been studied in As₂₀S₈₀ and Sb₂S₃ films, photodarkening in As₅₀Se₅₀ films, and As₂Se₃ has exhibited various photomechanical properties. Applications for chalcogenide glasses include phase change memory, solar cells and infrared waveguides and sensors. Biotic fabrication of such materials can produce vast quantities of nanostructures in a scalable low temperature process.

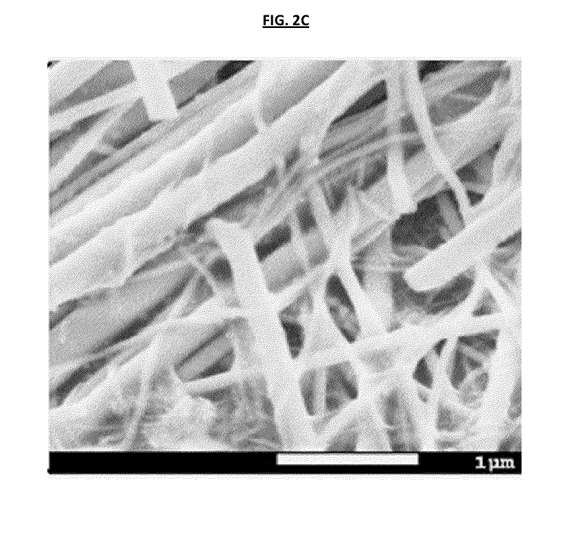

PbS can be produced with Torulopsis sp., CdS with E. coli, Fusarium oxysporum and Rhododpseudomonas palustris, ZnS from Rhodobacter spaeroides, and As₂Se₃ from Shewanella HN-41. Work using Shewanella strain HN-41 and Shewanella sp. Strain ANA-3 demonstrates the concomitant reduction of As(V) and S₂O₃²⁻ in solution to synthesize macroscopic quantities of inorganic AsₓS_{y} nanofibers. The resulting nanomaterials exhibit novel morphologies as shown in FIG. 4 that cannot easily be duplicated using known abiotic routes.

Bacterial Growth Methods

In one example, all biological cultures were prepared in lysogeny broth (LB), which served as a nutrient rich medium, until the optical density (OD) was between 1.5 and 2 absorption units (which correspond to 1.2E10 to 1.6E10 cells/ml). Cells were then inoculated into a chemically defined anaerobic minimal medium (MM) for experimentation.

The inocula were grown aerobically in 20 mL of LB medium from a frozen (−80° C.) stock, up to an optical density at 600 nm (OD₆₀₀) of 1.5±0.15. These aerobic pre-cultures were inoculated at 0.1% (vol/vol) into anaerobic serum bottles each containing 80 mL HEPES-buffered (30 mM) medium consisting of: 20 mM sodium DL-lactate, as electron donor; 28 mM ammonium chloride; 1.34 mM potassium chloride; 4.35 mM sodium phosphate monobasic; 20 mM sodium hydroxide; 10 mM thiosulfate, as Na₂S₂O₃.5H₂O; 5 mM arsenate, as Na₂HAsO₄.7H₂O. Vitamins, amino acids, and trace mineral stock solutions were used to supplement the medium. The medium was adjusted to an initial pH of 7.25, and anaerobic conditions were reached by purging with 100% N₂. The anaerobic serum bottles, sealed with butyl stoppers and aluminum seals, were sterilized by autoclaving at 120° C. for 15 minutes. Arsenate, thiosulfate, and vitamins were added after autoclaving. All cultures were grown at 30° C. and agitated at a rate of 150 rpm.

A general process for producing nanofibers includes inoculating a Shewanella sp. strain ANA-3 with a carbon and energy source (acting as an electron donor), a sulfur source and an arsenic source (acting as electron acceptors). The Shewanella sp. strain ANA-3 is grown. The resulting AsₓS_{y} nanofibers are harvested after a predetermined time.

The Shewanella sp. strain ANA-3 may grow on various carbon sources and electron donors. For example, the ANA-3 strain is able to grow on Lactate, Acetate (only anaerobically), and Pyruvate acting as carbon sources and electron donors while 5 mM arsenate may be used as the terminal electron acceptor. Other electron donors may include Citrate, Ethanol, Formate, Fumarate, Glucose, Glycerol, Malate, Propionate, and Succinate. Other electron acceptors may include Oxygen, Fumerate, Selenate, Nitrate, MnO₂, Fe(OH)₃, AQDS, Sulfate, Thiosulfate, Sulfite and DMSO.

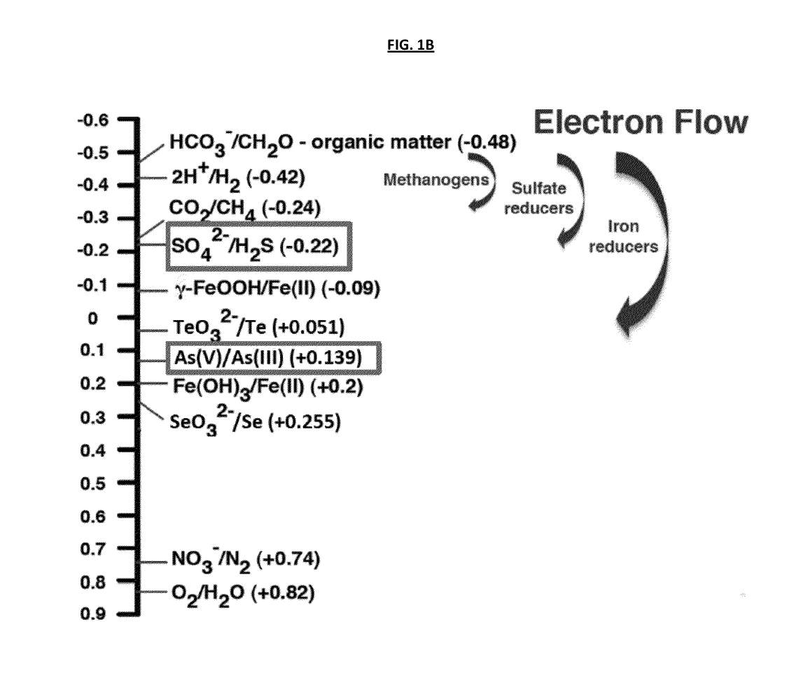

Specifically, a Shewanella sp. strain ANA-3 was grown anaerobically in MM containing 20 mM lactate, 10 mM thiosulfate, and 5 mM arsenate. During incubation, within 18 to 20 hours after inoculation, macroscopic quantities of AsₓS_{y} nanofibers began to form. FIG. 2A illustrates precipitated arsenic sulfide after several days according to an embodiment. FIG. 2B illustrates an SEM image of arsenic sulfide according to an embodiment. FIG. 2C illustrates arsenic sulfide as a mesh of polydisperse fibers according to an embodiment. It appears that Shewanella oxidized lactate as the electron donor and reduced thiosulfate as an electron acceptor for respiration, while also reducing As(V) to As(III), results in sulfur and arsenic compounds subsequently precipated as AsₓS_{y} nanostructures.

Morphological, Compositional and Crystallographic Properties of Biogenic Fibers

Precipitated AsₓS_{y} observed by scanning electron microscope (SEM) showed the material to be polydisperse fibers ranging from 20 to 600 nm in diameter and from 0.1 to 100 μm in length. Energy-dispersive X-ray Spectroscopy (EDS) confirmed the fibers to be made of arsenic and sulfur and Electron Microprobe Analysis (EMPA) found the average ratio to be 38:62 which corresponds within 5% to that of As₂S₃.

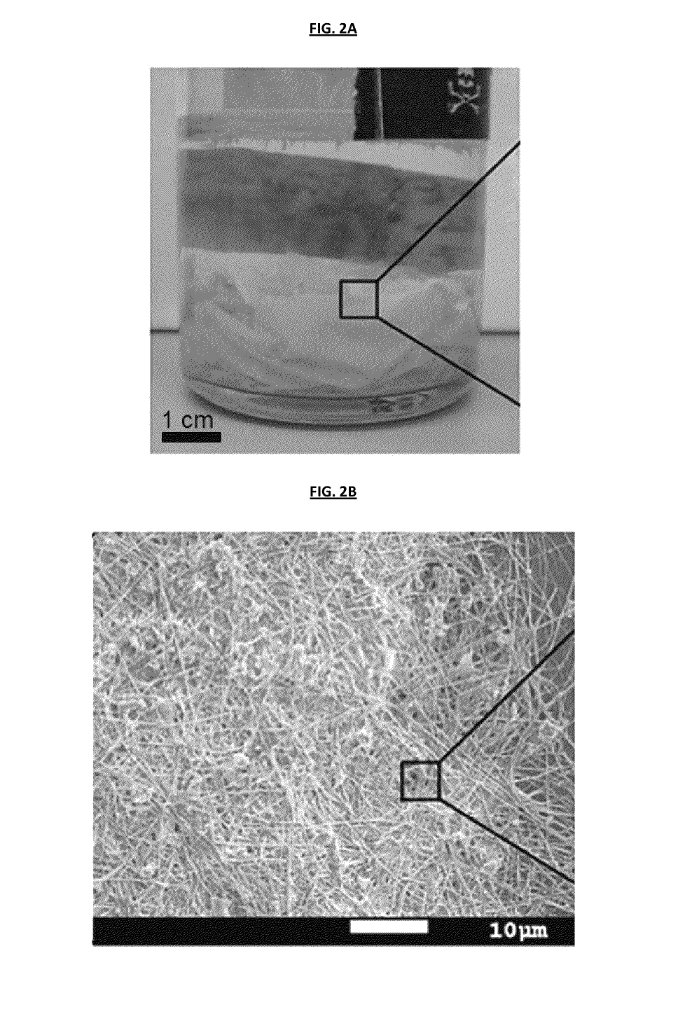

FIG. 3A illustrates an SEM image of a fiber used for the EDS composition measurement according to an embodiment. FIG. 3B illustrates EDS data showing the fiber to be made of arsenic sulfide according to an embodiment. High silicon and oxygen peaks are due to the SiO₂ substrate beneath it.

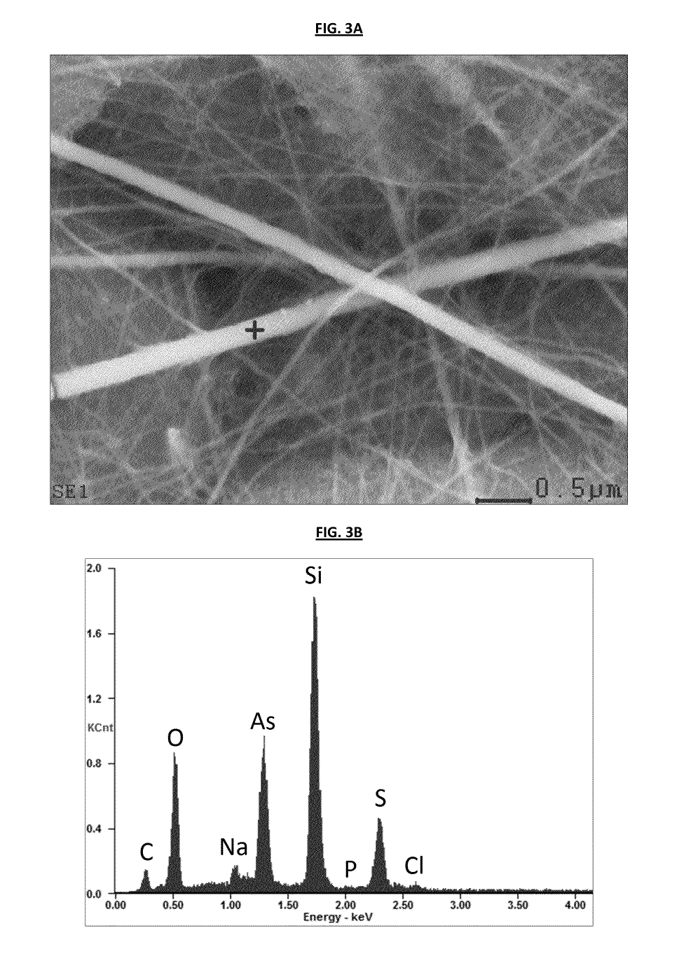

Some fiber morphologies suggest that the material possesses a crystalline structure as illustrated by FIG. 4. FIG. 5A is an SEM image of a fiber fabricated according to the process described above with branches coming off at similar angles. Repeated angles and planes suggest a uniform crystal structure. FIG. 5B illustrates batch X-Ray Diffraction (XRD) of a fiber fabricated according to the process described above, showing distinct peaks representing β-As₄S₄, also referred to as As₈S₈. Wide peaks suggest crystalline domains are small relative to the overall amorphous material. Diffraction patterns were attempted on a JEOL JEM-2100F transmission electron microscope (TEM), however the crystalline regions of the sample were too beam sensitive for analysis. β-As₄S₄ has been reported to be extremely beam sensitive, also preventing diffraction analysis. The beam sensitivity of the sample supports the sample's identification as β-As₄S₄.

Optical Properties

Average band gap of the fibers was calculated fitting UV-Vis spectra to the equation:

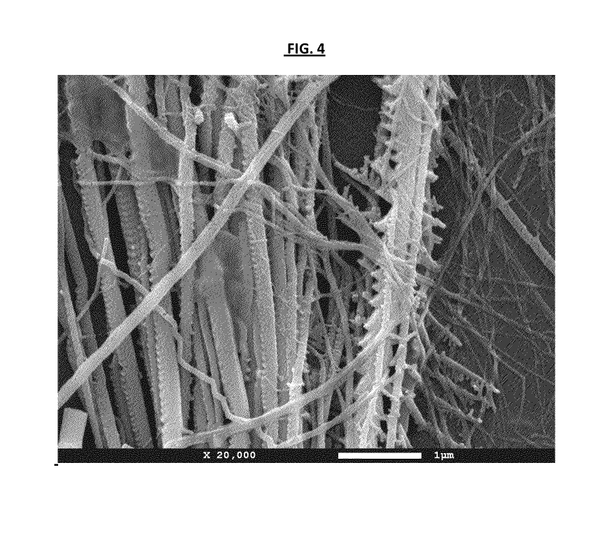

(ahv)ⁿ=A(hv−E_{g})

where a is the absorption (in arbitrary units), hv is the energy of the incident photon, n is an exponential constant that depends on the type of the band gap (n=2 or ½ for direct and indirect band gaps, respectively), A is a scaling factor, and E_{g} is the band gap energy. Fitting the linear region of the UV-Vis data to the above equation and extrapolating the fit to the abscissa axis gives the band gap energy E_{g}=hv. FIG. 6 is a “Tauc Plot” of washed AsₓS_{y} fibers according to an embodiment. Various runs produced band gaps ranging from 2.2 to 2.5 eV. This compares well to amorphous As₂S₃ which has an indirect band gap between 2.35 and 2.45 eV as opposed to roughly 2.6 eV reported for crystalline As₂S₃.

Identifying the Arsenic Reduction Pathway: Arsenic Operons of ANA-3

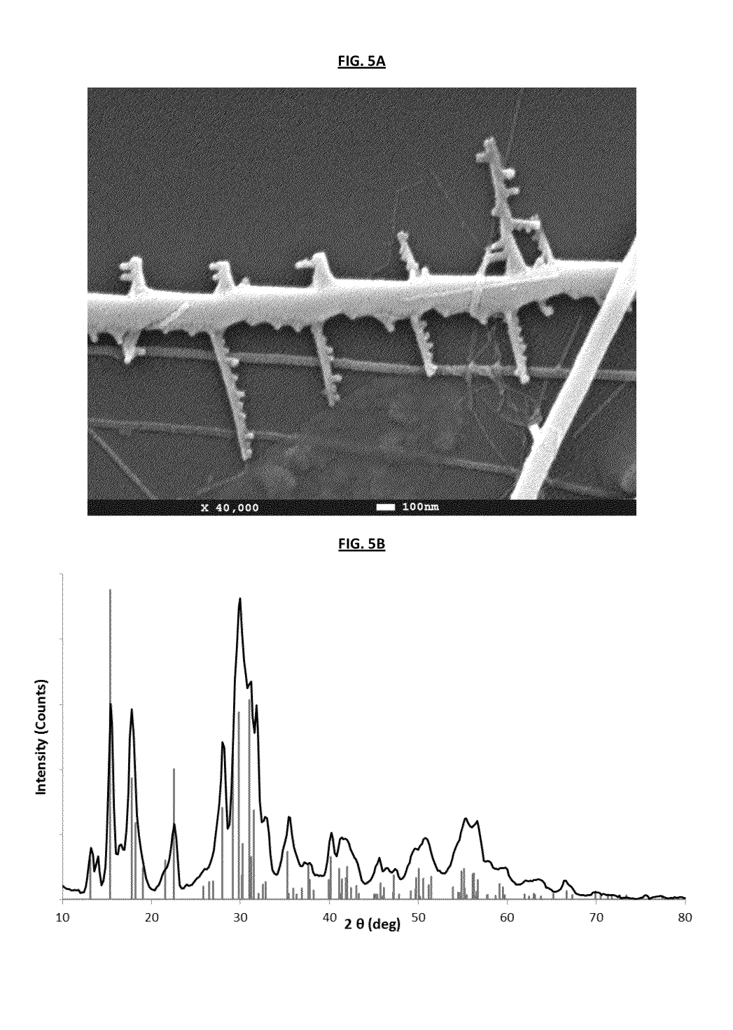

Shewanella sp. strain ANA-3 was used because it is a prodigious sulfur reducer and has two well understood arsenic related operons. FIG. 7 illustrates a map of the arr operon responsible for respiring arsenic and the ars operon responsible for detoxifying arsenic inside the cell according to an embodiment. The arr operon is responsible for reducing arsenic as part of the metabolic process, while the ars operon is responsible for reduction as a means of detoxification. The arr operon contains two genes: arrA and arrB. The protein ArrA is thought to be the reductase while ArrB is thought to act as a link in the metabolic chain connecting to c-type cytochromes. ANA-3 cannot use As(V) as an electron acceptor for respiration when missing either of these two genes. The ars operon contains five genes of interest: arsC, arsB, arsA, arsR (not shown), and arsD. ArsC is the reductase, ArsB is a cytoplasmic efflux pump to expel the reduced As(III), ArsA is an ATPase subunit that assists the ArsB pump, and ArsR and ArsD are regulators to control expression. The only known arsenic reductases are ArsC and ArrA for detoxification and respiration, respectively.

Four strains of ANA-3 were used to investigate the arsenic reduction pathway responsible for AsₓS_{y} production: ANA-3 wild type (ANA-3 WT), ANA-3 arrA mutant missing the respiratory reductase ArrA (ARRA3), ANA-3 arsB mutant missing the detoxification pump ArsB (ARSB1) and ANA-3 arsC and arrA mutant missing both reductases ArsC and ArrA (ARM1). When inoculated into MM with initial concentrations of 20 mM lactate, 10 mM thiosulfate and 5 mM arsenate, they all produced macroscopic quantities of arsenic sulfide material within 18 and 85 hours. Arsenic sulfide material made by each mutant was imaged using SEM showing all were in the form of nanofibers of similar dimensions (data not shown).

Table 1 below shows a list of strains used in this research alongside the delay between inoculation and the production of macroscopically visible quantities of AsₓS_{y}.

TABLE 1

| | | | Hours |
|---|---|---|---|
| Strain | Deletion | Description | to AsS production |
| ANA-3 | | Wild type | 19 ± 1 |
| ARRA3 | ΔarrA | lacks respiration reductase | 75 ± 10 |
| ARSB1 | ΔarsB | lacks detoxification pump | 38 ± 5 |
| ARM1 | ΔarrA/ΔarsC | lacks both respiration and | 38 ± 5 |
| | | detoxification reductases | |

ARM1′s ability to produce AsₓS_{y} suggests that either arsenic is being reduced abiotically or there is an additional pathway by which the cells can reduce arsenic.

Arsenic Reduction is Achieved Biotically

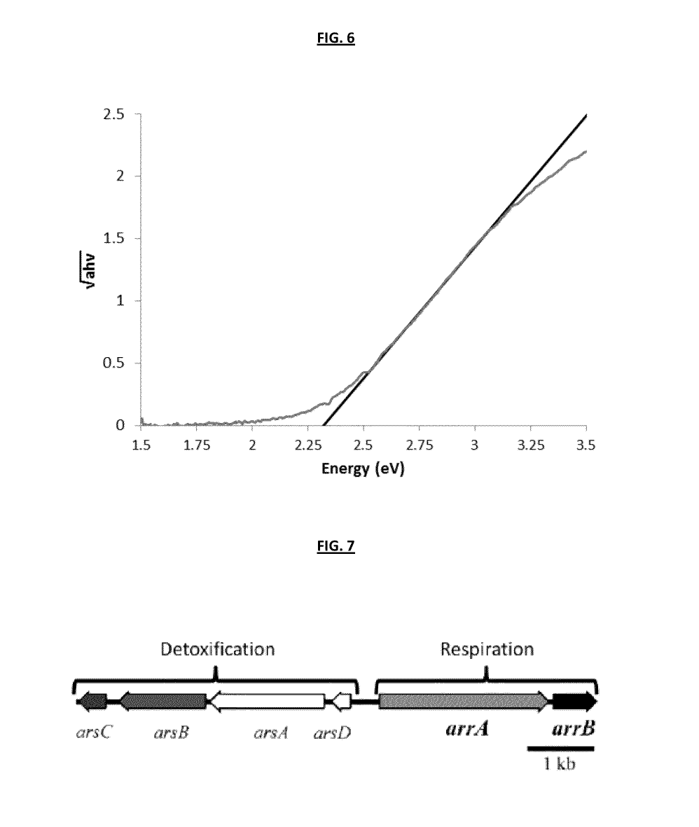

To test for abiotic arsenic reduction, ANA-3 WT was inoculated into MM with 20 mM lactate and 10 mM thiosulfate, but no arsenic. The culture developed for five days providing the bottles with reduced sulfur. The cultures were then heat killed (by heating to 55° C. oven for two hours) and injected with 5 mM of an either unreduced arsenic, As(V), or reduced arsenic, As(III). Thus, both bottles contained dead cells and reduced sulfur when injected with arsenic precursors. FIGS. 8A and 8B illustrate the results wherein the left bottle contains unreduced As(V) while the right contains reduced As(III). The As(III) bottles turned a pale yellow within 5 minutes as shown in FIG. 8A and formed visible arsenic sulfide material over 30 days as shown in FIG. 8B. The As(V) bottles remained clear for the entirety of the experiment, never forming visible concentrations of AsₓS_{y} compounds.

The inability of the As(V) bottles to produce coloration resembling the arsenite bottles over long time spans strongly indicates that dead cells (and the remnants thereof) cannot reduce As(V) to As(III). This data in conjunction with the mutant screening strongly suggests that there is an alternate biotic pathway to arsenic reduction that is independent of the ArrA and ArsC reductases. One hypothesis is that ARM1 utilizes outer cytochromes, such as ones similar to OmcA and MtrC, to reduce As(V) extracellularly similar to how Shewanella MR-1 reduces Se(IV) and Fe(III). For instance, CymA, the protein at the base of the direct electron transfer pathway ending with OmcA/MtrC, is needed in addition to ArrA and ArrB for As(V) reduction. Such extracellular reduction would not necessitate toxic As(V) entering the cell where it may do damage. This pathway may explain why MR-1 can produce As—S fibers at all, despite not having any genes that match the As reductases ArrA or ArsC to any high degree of similarity.

Another possibility is that As(V) is being reduced similarly to several proposed S₀ reduction pathways revolving around an intraspecies sulfur cycle. In this system, thiosulfate S₂O₃²⁻ is reduced to HS⁻ and SO₃²⁻. One, or both, of the products then reacts with As(V) to produce As(III). The resulting oxidized sulfur is then able to be reduced again by cellular metabolism. This cycle, or one similar, may be responsible for the apparent As(V) reduction by the double deletion mutant, ARM1.

Nanofiber Growth Investigation: Demonstration of Abiotic Nanofiber Extension

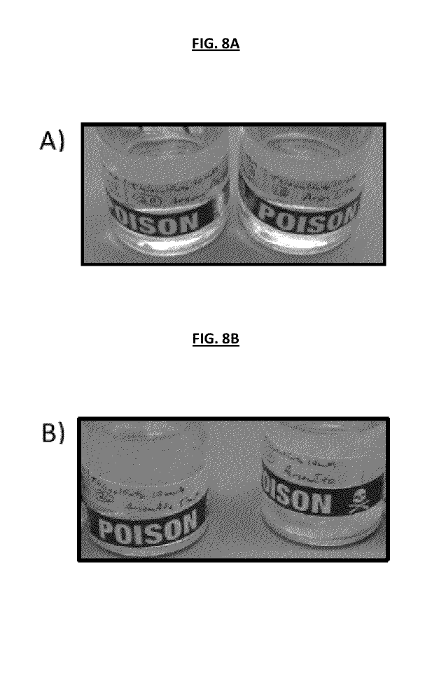

To understand the role of the bacteria in fiber extension, a typical ANA-3 experiment was interrupted while the fibers were still extending. Two cultures were heat killed after 42 hours of normal cell growth and fiber production. The OD continued to rise after death indicating an increase in particulate AsₓS_{y}. Thus, once formation has begun, fiber extension can proceed abiotically. FIG. 9 illustrates an OD reading of two heat killed cultures and one control according to an embodiment. Heating occurred between 42 and 44 hours. Death did not halt fiber extension indicating extension is potentially an abiotic process.

In Vivo Measurements of Fiber Growth

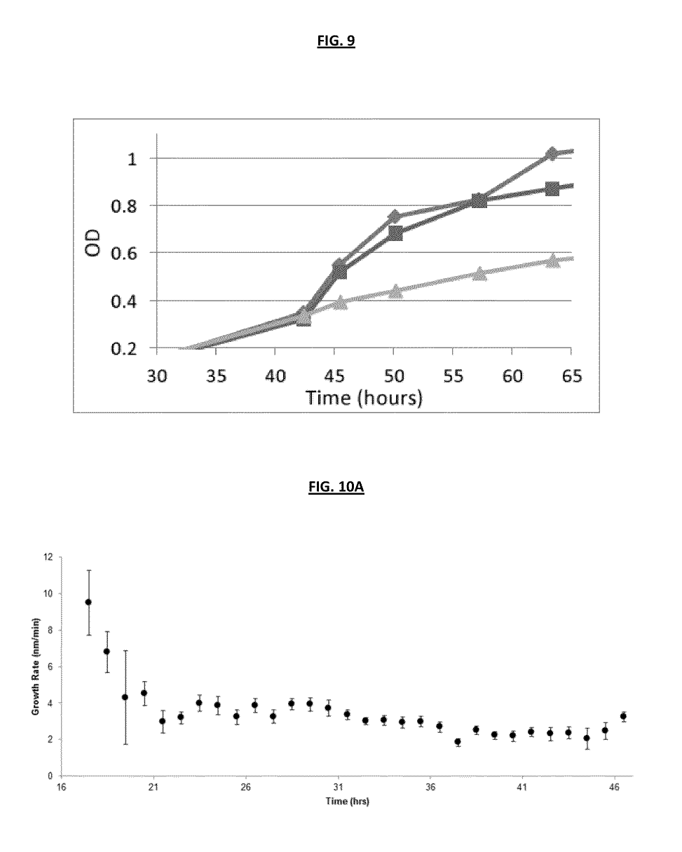

Direct in vivo observation of fiber growth was done using an inverted Nikon Ti light microscope taking time-lapse images every five minutes. “Perfect focus” feature allowed videos lasting several days to be collected. Analysis of the time-lapse images determined the average rate of fiber extension at room temperature to be 3.8 nm/min, starting as high as 10 nm/min and slowing over time as shown in FIG. 10A. FIG. 10A illustrates average fiber growth rate versus time according to an embodiment. The growth rate starts high and decreases to a steady rate. This behavior is highly repeatable.

Several videos demonstrated that fiber extension is occurring at the tip of the fiber rather than the base as shown in FIG. 10B. FIG. 10B illustrates time-lapse snapshots of extending fibers according to an embodiment. The time-lapse snapshots of extending fibers shows a kink appear in the third frame and the fiber continuing to extend from the kink in the fourth frame.

A non-specific protein stain (NanoOrange) was added to check for proteins along the length of the fibers that may act as scaffolds or leads as shown in FIG. 10C. FIG. 10C illustrates NanoOrange protein stain of a cluster of As—S fibers according to an embodiment. There are no signs of proteins along the length of the fibers. These results together suggest that once fiber growth has begun, ANA-3 plays little role in the extension of the fibers. Remaining roles the cells may be filling are sulfur and arsenic reduction and potentially providing nucleation sites to initiate fiber growth.

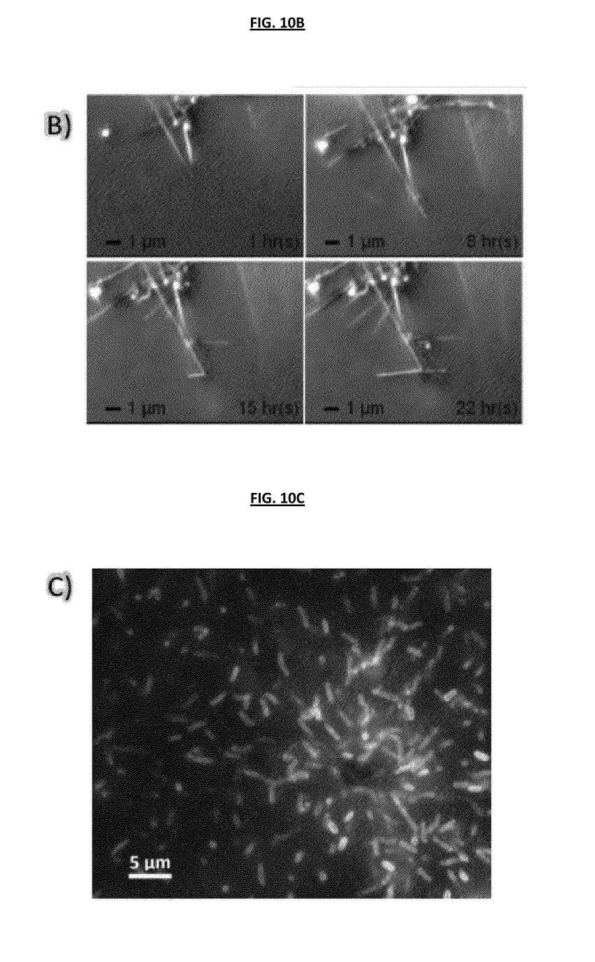

FIG. 11 illustrates growth curves of four ANA-3 strains showing a local maximum in cell density just before arsenic sulfide production and then a drastic death phase as macroscopic quantities of the material are then produced. This direct correlation between cell density and nanofiber production indicates a strong biotic role in their formation and a weak role in their extension.

Determination of Nucleation/Seed Sites

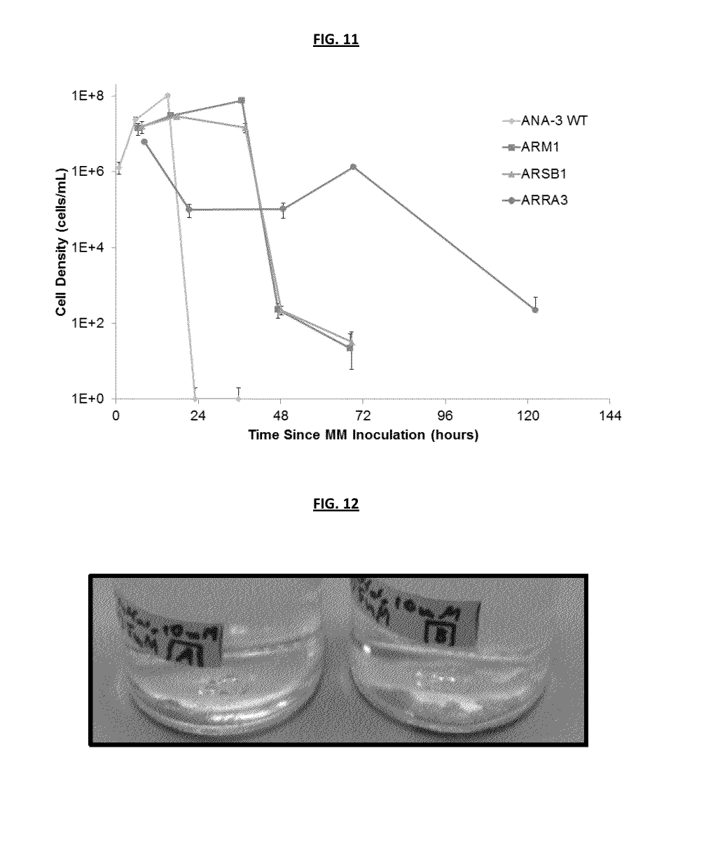

In vivo data has shown fibers growing from ANA-3, inanimate particulate matter, and empty sections of glass. This suggests that the nucleation sites are not restricted to the cells themselves. To verify, ANA-3 was inoculated into two MM bottles with 20 mM lactate and 10 mM thiosulfate but no arsenic. After 27 hours an estimated 99% of the original cell density was removed via filtration with a 220 nm filter. Remaining cells in both cultures were killed with kanamycin (50 and 100 μg/mL in filtered and unfiltered, respectively). The resulting bottles shown in FIG. 12 show both cultures produced macroscopic quantities of AsₓS_{y}. The filtered bottle A produced roughly 25% produced by the AsₓS_{y} of the unfiltered bottle B according to an embodiment. A 99% decrease in whole cells did not result in a similar decrease in AsₓS_{y} production. This supports the hypothesis that the cells are not the primary nucleation sites. This data also suggests that if a specific seed is needed, it is likely to be smaller than the filter size used, 220 nm.

Nanofiber Field Effect Transistor

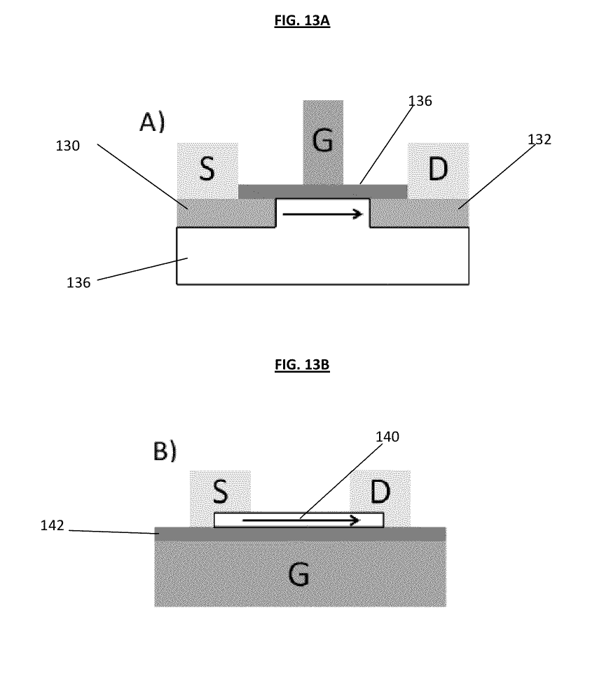

The Field Effect Transistor (FET) is an important device, both for its applications in electronics and as a tool to probe the properties of semiconductors. A MOSFET is a FET characterized by a meta-oxide-semiconductor (MOS) structure consistent of a metallic acting gate (usually highly doped polysilicon), an insulating oxide layer (usually SiO₂), and the underlying semiconductor as shown in FIG. 13A. FIG. 13A is a diagram of a typical MOSFET where S and D label the source and drain contact, respectively and G labels the gating contact. A source 130 and drain 132 are formed under the source contact S and drain contact D respectively. An insulating oxide layer 134 is formed under the gate contact G a region 136 is the semiconductor substrate. A p- or n-type doped substrate determines the type of MOSFET (either p-type or n-type). The current running through the gate-activated region of the semiconductor is shown by the arrow.

The above As—S nanofiber material may be applied to different semiconductor devices such as the MOSFET described above. The versatility of the As—S nanofiber material in relation to light leads to a wide range of tunable functionalities in various components including sensors, waveguides, photonic crystals, infrared and photoactive devices. In such devices, the nanofiber material may be used advantageously for the semiconductor material.

For example, fabrication and testing of an inverted MOSFET structure may be done where a biotically influenced AsₓS_{y} nanofiber fabricated according to the above mentioned process serves as the semiconducting material as shown in FIG. 13B. FIG. 13B is a diagram of an As—S nanofiber transistor according to one example. In FIG. 13B, S and D are patterned gold contacts, while G represents a charged brass chuck the chip is resting on and the lightly doped silicon substrate. A region 140 represents an As—S fiber that may be deposited on a thin oxide layer 142. The As—S nanofiber acts as the semiconductor material in this example.

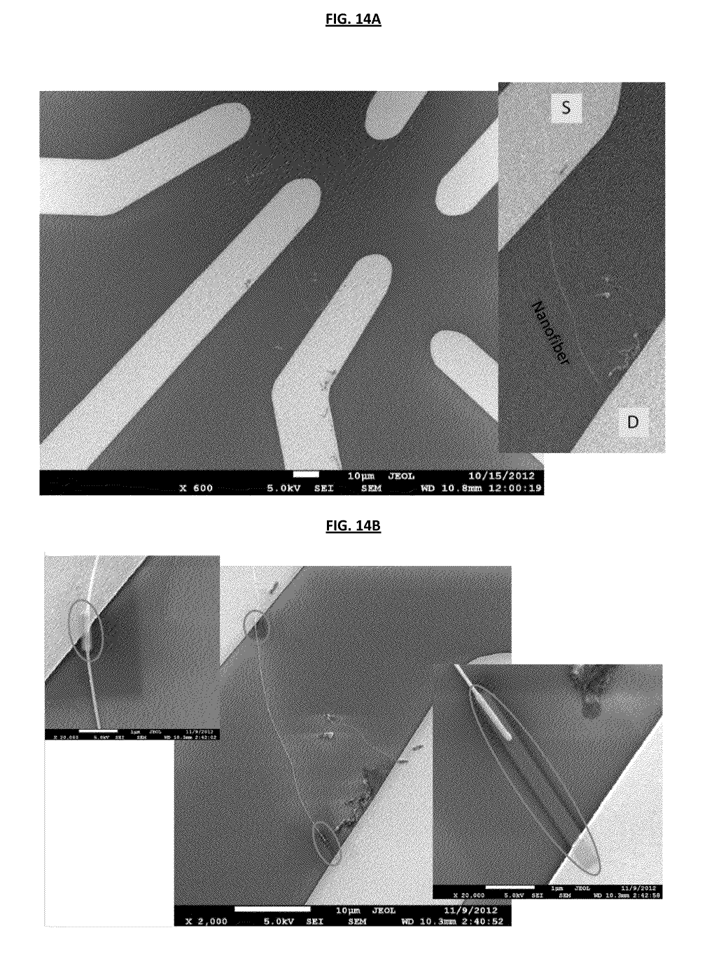

FIG. 14A illustrates a nanofiber before contact deposition between a source and a drain contact in a semiconductor device according to an embodiment. FIG. 14B illustrates a nanofiber after contact deposition where the nanofiber is in contact with the source and drain contact according to an embodiment.

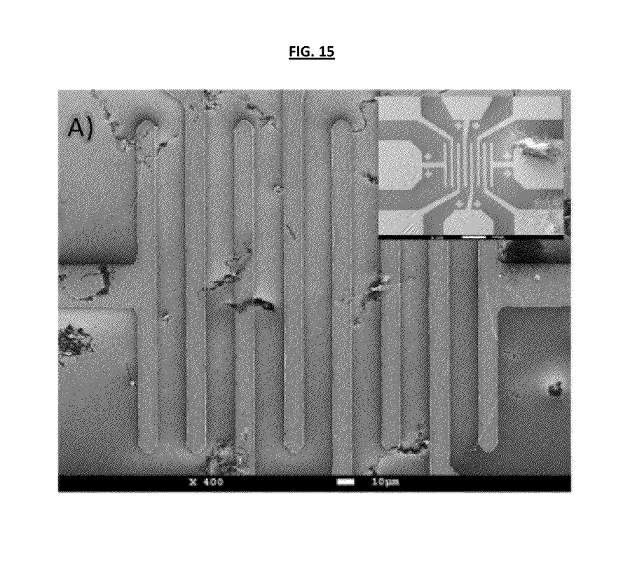

Fibers were drop deposited onto polydoped silicon chips with an SiO₂ layer topped with gold contacts as shown in FIG. 15. FIG. 15 is an SEM image of interleaved gold contacts with deposited As—S fibers according to an embodiment. The source-to-drain current, I_{SD}, was measured as a function of the bias voltage, V_{SD}, and gate voltage, V_{G}, using an Agilent 4156C Precision Semiconductor Parameter Analyzer probe station in this example.

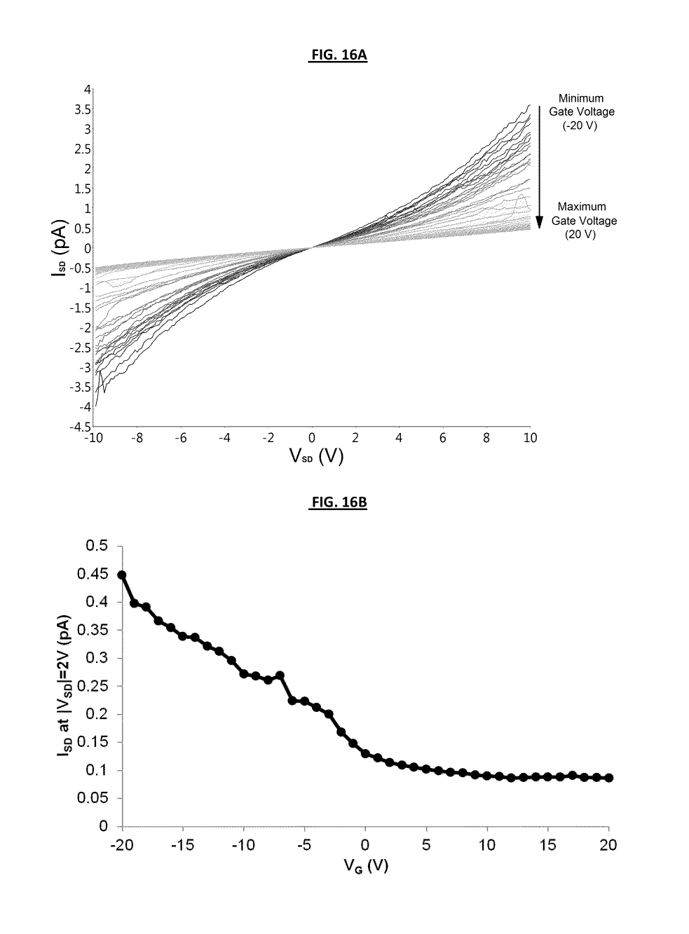

Nonlinear I_{SD}−V_{SD} curves shown in FIG. 16A and gating behavior (decreased resistance for negative gate voltages shown in FIG. 16B indicate the fibers in this device are p-typed semiconductors. FIG. 16A illustrates the I_{SD}−V_{SD} curves at back-gate voltages from −20 V to +20 V according to an embodiment. FIG. 16B illustrates gating behavior as current magnitude increases for negative back-gate voltage for a fixed source-drain voltage, at ±2 V, according to an embodiment.

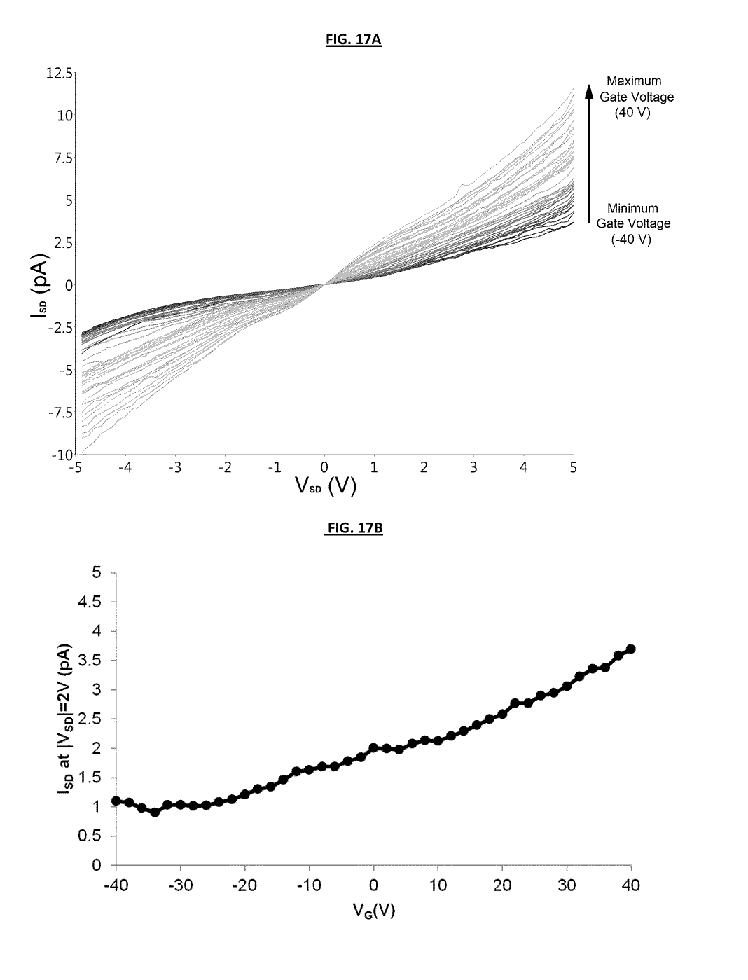

Nonlinear I_{SD}−V_{SD} curves shown in FIG. 17A and gating behavior (decreased resistance for positive gate voltages) shown in FIG. 17B indicate the fibers in this device are n-typed semiconductors. FIG. 17A illustrates the I_{SD}−V_{SD} curves at back-gate voltages from −40 V to +40 V according to an embodiment. FIG. 17B illustrates gating behavior as current magnitude increases for negative back-gate voltage for a fixed source-drain voltage, at ±2 V, according to an embodiment.

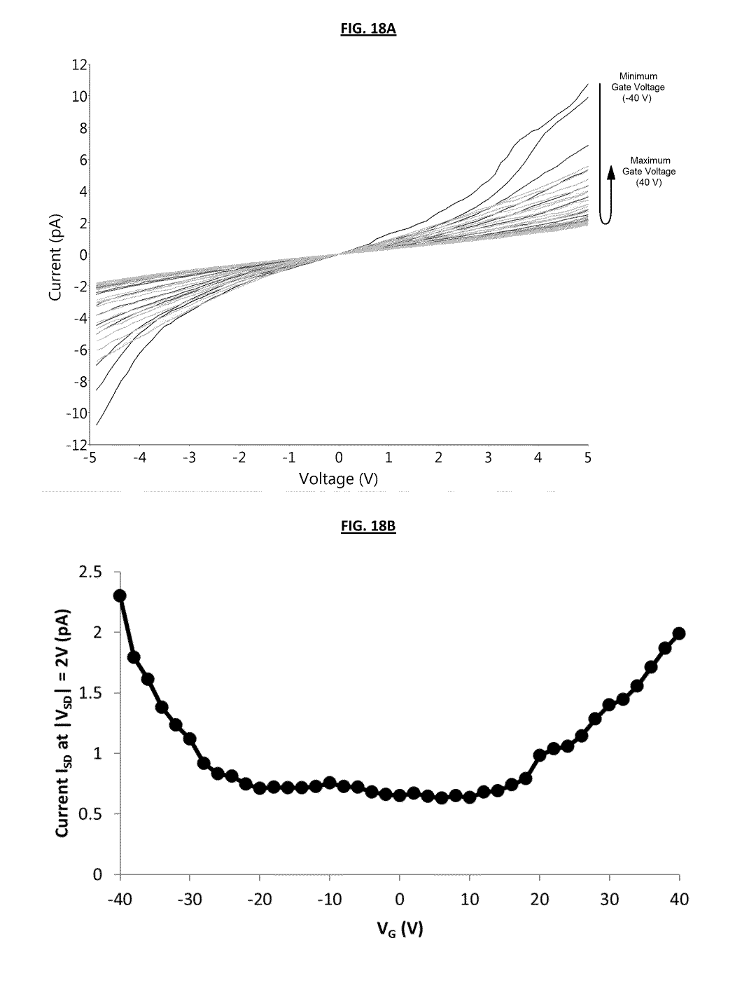

62% of fabricated devices were P-type, 23% were N-type, and 15% were too complex to fit clearly as one type. For example, FIG. 18A illustrates the I_{SD}−V_{SD} curves at back-gate voltages from −40 V to +40 V and FIG. 18B illustrates gating behavior as current magnitude increases for negative back-gate voltage for a fixed source-drain voltage, at ±2 V, of a nonlinear device such as the transistor in FIG. 13B made from a bundle of fibers that appears to contain at least one P-type and one N-type nanofiber. This device is thus a transistor that turns “on” for both positive and negative gate voltages.

The higher percentage of P-type fibers is thought to be due to carbon acting as a dopant among the dominant As₂S₃ glass. Deviations from (2,3) stoichiometry may also lead to P-type fibers (As_{2+X}S₃) or N-type fibers (As₂S_{3+X}). The effect of the present crystalline β-As₄S₄ is unknown, though from a purely stoichiometric standpoint β-As₄S₄ would increase the ratio of arsenic, suggesting P-type behavior.

Regardless of physical geometry, the devices have similar properties to traditional MOSFETs, such as the threshold voltage and linear mobility. More AsₓS_{y} nanofiber FET devices can be constructed and characterized to provide average values of these properties.

Measure the Optomechanical Effect in Nanostructures

The optomechanical properties of As₄₀Se₆₀ and As₄₀S₆₀ amorphous films have been reported. No work, as of yet, has been published exploring the optomechanical properties of As₂S₃ when formed as a quai-1D nanostructure. In the optical microscope, several fibers can be observed exhibiting plastic deformation upon exposure to high intensity non-filtered light. The characterization of this effect for biotically influenced nanofibers is new information on this property.

Conclusion

The biological route proposed is scalable via large-volume bioreactors and offers a unique approach to combinatorial materials science by taking advantage of comparative genomic analyses to discover microorganisms capable of processing different metals and chalcogens. The targeted materials have the potential to serve energy-conversion needs in photovoltaic solar cells and micro-opto-mechanical systems. The optomechanical effect, in particular, can be used for all-optical actuation in flammable and aqueous environments where the use of high voltages (e.g., piezoelectric devices) would be hazardous.

For the disclosed model system of ANA-3 and AsₓS_{y}, it has been demonstrated that arsenic reduction is being done by the cell, but not by using either of the two known reductases. The inventors have verified reported properties such as composition and diverse general morphology. The inventors have also measured several new properties of the fibers, such as growth rate, primary crystalline phase, band gap, and semiconducting type. The nature of nanofiber nucleation points and single fiber crystal structure will continue to be investigated. Fabrication of nanofiber FET devices and optomechanical studies hold the potential to be both illuminating and applicable to the nanotech industry.

Embodiments have been described in relation to particular examples, which are intended in all respects to be illustrative rather than restrictive. Further, while embodiments have been described in connection with a number of examples and implementations, it is understood that various modifications and equivalent arrangements can be made to the examples while remaining within the scope of the inventive embodiments.

Other embodiments will be apparent to those skilled in the art from consideration of the specification and practice of the embodiments disclosed herein. Various aspects and/or components of the described embodiments may be used singly or in any combination. It is intended that the specification and examples be considered as exemplary only, with a true scope and spirit of the disclosure being indicated by the following claims.

What is claimed is:

1. A method for production of nanotubes based on arsenic-sulfide (As—S) compounds including As₂S₃ by reacting thiosulfate with arsenate through mediation of Shewanella sp. strain ANA-3.

2. A method of producing nanotubes comprising:
inoculating a Shewanella sp. strain ANA-3 with a carbon and energy source, a sulfur source and an arsenic source;
growing the Shewanella sp. strain ANA-3; and
harvesting resulting AsₓS_{y} nanofibers after a predetermined time.

3. The method of producing nanotubes of claim 2, wherein the predetermined time is between 18 and 72 hours

4. The method of claim 2, wherein the nanofibers include amorphous As₂S₃

5. The method of claim 2 wherein the carbon and energy source is one of the group of Acetate, Lactate, or Pyruvate.

6. The method of claim 2, wherein the sulfur source is thiosulfate and the arsenic source is arsenate.

7. The method of claim 2, wherein the nanofibers include nanofibers having a crystalline structure.

8. The method of claim 2, wherein the nanofibers include crystalline β-As₄S₄.

9. The method of claim 2, wherein the Shewanella sp. strain ANA-3 includes reductase ArsC and ArrA.

10. An AsₓS_{y} nanofibers nanotube compound produced by inoculating a Shewanella sp. strain ANA-3 with a carbon and energy source, a sulfur source and an arsenic source and harvesting resulting AsₓS_{y} nanofibers after a predetermined time.

11. The nanotube compound of claim 10, wherein the nanofibers include amorphous As₂S₃

12. The nanotube compound of claim 10, wherein the carbon and energy source is one of the group of Acetate, Lactate, or Pyruvate.

13. The nanotube compound of claim 10, wherein the sulfur source is thiosulfate and the arsenic source is arsenate.

14. The nanotube compound of claim 10, wherein the nanofibers include nanofibers having a crystalline structure.

15. The nanotube compound of claim 10, wherein the nanofibers include crystalline β-As₄S₄.

16. The nanotube compound of claim 10, wherein the Shewanella sp. strain ANA-3 includes reductase ArsC and ArrA.

17. A semiconductor device comprising:
a substrate,
a semiconductor including nanofibers composed of AsₓS_{y} deposited on the substrate;
wherein the nanofibers are formed by inoculating a Shewanella sp. strain ANA-3 with a carbon and energy source, a sulfur source and an arsenic source and harvesting resulting AsₓS_{y} nanofibers after a predetermined time.

18. The semiconductor device of claim 17, further comprising:
a source region;
a drain region; and
wherein the semiconductor is in contact with the source region and the drain region.

19. The semiconductor device of claim 17, wherein the device is a solar cell.
